(19) United States
(12) Patent Application Publication
(10) Pub. No.: US 2017/0181960 A1
(43) Pub. Date: Jun. 29, 2017

(54) NATURAL OIL NAIL COATING REMOVAL COMPOSITIONS

(71) Applicant: JAMBERRY NAILS LLC, American Fork, UT (US)

(72) Inventor: Douglas Dean Schoon, Dana Point, CA (US)

(21) Appl. No.: 15/164,456

(22) Filed: May 25, 2016

Related U.S. Application Data
(60) Provisional application No. 62/272,514, filed on Dec. 29, 2015.

(51) Int. Cl. A61K 8/92; A61Q 3/04

(52) U.S. Cl. CPC A61K 8/922; A61Q 3/04

(57) ABSTRACT

A nail coating removal composition includes one or more natural, plant-based oils formulated to condition, regenerate, and protect a nail plate, and one or more natural oils formulated to promote removal of a nail coating from the nail plate upon contact with the nail plate. The nail coating removal composition is substantially free of volatile solvents such as short-chain alcohols, ketones, and alkyl esters of organic acids.

### CROSS-REFERENCE TO RELATED APPLICATIONS

This application claims the benefit of U.S. Provisional Patent Application No. 62/272,514, filed on Dec. 29, 2015, the disclosure of which is incorporated herein in its entirety.

### BACKGROUND

The present disclosure relates generally to compositions and methods for removing cosmetic or medical fingernail and toenail coatings, including coverings, films, wraps (a fabric held to the nail plate using a cyanoacrylate adhesive), polishes, adhesives, decorative self-adhesive strips (using pressure-sensitive and/or thermal-sensitive adhesive) such as shields, stickers and decals; and other products for fingernails and toenails.

While beneficial, use of such nail coatings is commonly associated with damage to a wearer's nail plate. In particular, removal of such nail coatings (often involving pulling and stretching of adhesive remnants) can result in lifting and delaminating of upper layer cells of the nail plate, which causes detrimental and unwanted thinning of the nail plate.

In addition, compositions for removing nail polish from a nail or for loosening a nail coating from a nail plate are typically formulated with acetone or other harsh ketones as the major active ingredient. Such compositions are generally considered to provide acceptable removal of the nail coating; however, use of the compositions is also associated with drying and other damage to the nail plate, and skin folds surrounding the nail plate. Various components included within traditional removal compositions are known to penetrate into the nail plate, contributing to drying out of the keratin and defatting and dehydrating the nail plate, leading to brittle nails, a thinner and/or pitted nail plate, and a weaker nail edge, among other detrimental effects.

Further, typical acetone-based compositions suffer from high flammability and high volatility, and are also known to suffer from a characteristic, pungent odor that many find to be unpleasant. Although attempts have been made to develop nail polish removal compositions that reduce these undesirable effects, there exists an ongoing need for compositions that provide acceptable removal without excessive damage to the nail plate and to surrounding tissue.

In addition, typical compositions are formulated specifically for removing pigmented lacquers and enamels (most commonly referred to as “polish”), and not for the removal of nail coatings having a solid structure, such as strips, wraps, or films that are adhesively applied to the nail plate. Although some nail polish removal compositions may be used for removing such solid nail coatings, traditional removal compositions can require long exposure times and can exacerbate the detrimental effects to the nail and surrounding tissues. In addition, wearers of such nail coatings may seek to minimize these unpleasant effects by limiting exposure time and prematurely forcing the covering off of the nail, leading to further damage to the nail plate and particularly to the edge of the nail plate.

### BRIEF SUMMARY

Certain embodiments described herein relate to a nail coating removal composition, comprising a blend of natural oils formulated to promote removal of a nail coating from a nail plate upon contact with the nail coating and formulated to condition the nail plate, wherein the removal composition is substantially free of volatile solvents, such as ketones, esters, alkyl esters of organic acids, and other oil dissolving substances.

It has been found that nail coating removal compositions of the present disclosure that reduce or eliminate volatile solvent and volatile solvent-like substances are able to beneficially protect the nail plate, aid in the regeneration of damaged nail plates and other nail tissue, and provide the nail plate with greater resistance to future damage. In addition, when a nail coating removal compositions is formulated according to one or more embodiments disclosed herein, the removal composition can provide such benefits to nail tissues while also functioning to effectively remove the nail coating.

In some embodiments, a nail coating removal composition includes one or more C8 to C18 free fatty acids and/or C8 to C18 fatty acid alcohols, such as caprylic, capric, lauric, myristic, palmitic, stearic, palmitoleic, oleic, linoleic, linolenic, and stearidonic acid, and/or related fatty acid alcohols.

Some embodiments alternatively or additionally include plant-based natural oils such as cottonseed, hempseed, pumpkinseed, flaxseed, grapeseed, coconut, avocado, argan, babussu, corn, safflower, poppyseed, sunflower, walnut, soya bean, canola, chia seed, perilla seed, palm kernel, peanut, palm, jojoba, macadamia nut, sesame seed, meadowfoam seed, wheat germ, and olive oil.

In preferred embodiments, the natural oil blend is formed of one or more of cottonseed oil, flaxseed oil, hempseed oil, chia seed oil, pumpkinseed oil, grapeseed oil, perilla seed oil, wheat germ oil, safflower oil, and poppyseed oil. Some embodiments include a natural oil in combination with a caprylic acid, myristic acid, myristic alcohol, cetyl alcohol, stearyl alcohol, stearic acid, oleic acid linoleic acid, and/or lauric acid component, which may be added to make up about 0.5 to 40%, or about 1 to 30%, or about 2 to 20% of the overall composition (e.g., may be added to one or more of the foregoing natural oils).

In some embodiments, the composition has a high concentration of C12 to C18 fatty acids and/or fatty acid alcohols. In preferred embodiments, the composition has a high concentration of C16 to C18 fatty acids and/or fatty acid alcohols, and more particularly a high concentration of C18 fatty acids and/or fatty acid alcohols. In one example, the composition may be formulated with a fatty acid profile of about 90% or more, or about 95% or more, or about 97% or more of C12 to C18 fatty acids and/or fatty acid alcohols. In another example, the composition is formulated with a fatty acid profile of about 90% or more, or about 93% or more, or about 95% or more, or about 97% or more of C16 to C18 fatty acids and/or fatty acid alcohols. In another example, the composition is formulated with a fatty acid profile of about 60-95%, or about 65-90%, or about 70-85% of C18 fatty acids and/or fatty acid alcohols. In some embodiments, the composition has a C18 fatty acid profile of greater than about 65%, 70%, 75%, 80%, 85%, 90%, or 95%.

In some embodiments, the amount of fatty acid alkyl esters is less than 50%, less than 25%, less than 10%, less than 5%, or less than 1% by weight of the composition. In some embodiments, substances insoluble in the removal composition at room temperature are omitted. In some embodiments, a composition has less than 30%, less than 20%, less than 10%, less than 5%, less than 2.5%, less than 1%, less than 0.1%, or less than 0.01% by weight of short-chain alcohols, ketones, alkyl esters of organic acids, or other volatile solvents.

In some embodiments, a method of removing a nail coating from a nail plate of a wearer includes: (1) providing a nail coating removal composition, the nail coating removal composition including one or more natural oils and being substantially free of volatile solvents; (2) contacting the nail coating removal composition to a nail coating; and (3) removing the nail coating from the nail plate.

In some embodiments, a method of removing a nail coating from a nail plate further includes, after contacting the nail coating removal composition to the nail coating, allowing the nail coating removal composition to disrupt at least one of the cohesive integrity of the nail coating and/or the adhesive bond between the nail coating and the nail plate. In some embodiments, the nail coating is a solid nail coating, such as a nail strip, shield, wrap or film, and the nail coating removal composition enables removal by disrupting the cohesive integrity of the nail coating itself and/or by disrupting the adhesive bond between the nail coating and the nail plate (e.g., through wicking action of one or more of the natural oils included in the composition).

### DETAILED DESCRIPTION

### Definitions

As used herein, the terms “nail,” “nail plate,” “cuticle,” “nail folds,” “nail bed,” “nail edge,” and other anatomical terms referring to the nail and surrounding tissues refer to fingernails and toenails. Accordingly, description having a specific reference to a fingernail in a particular embodiment should be understood to apply, in other embodiments, to a toenail as well, and vice versa.

As used herein, the terms “nail cover,” “nail covering,” “nail coating,” and similar terms refer to an article that is coated, positioned, or layered onto a nail plate for one or more cosmetic, decorative, medical, or other functional purposes. The term “nail coating” and similar terms include liquid compositions that cure after application to the nail plate, such as common nail polishes. Such compositions may be referred to herein as “liquid nail coatings.” It will be understood, however, that these compositions are only applied as liquids, and afterwards take on a solid form upon curing on the nail plate.

The term “nail coating” and similar terms also refer to a solid piece of material (e.g., a strip, shield, sticker, decal or film made from a vinyl polymer or from other materials) configured to be adhered to the nail plate to cover at least a portion of the nail plate. Such articles may be referred to herein as “solid nail coatings.” Although various terms are used in the art to refer to such solid nail coatings, one of skill in the art will understand that many solid nail coverings include an adhesive layer or are applied to a nail plate using an applied adhesive that allows the solid nail coating to be sealed to the nail plate using pressure or a combination of pressure and elevated temperatures. For example, a thermal-setting adhesive can be formulated to set and/or set more securely with the application of heat.

In one example, an adhesive suitable for use with a solid nail covering is an acrylate polymer, such as an acrylate(s)/ethylhexyl acrylate copolymer. Embodiments of removal compositions described herein have been shown to be particularly effective in removing thermal-setting and/or pressure-setting solid nail coverings applied using such adhesives. Exemplary solid nail coatings usable with the removal compositions disclosed herein include those nail coatings (including coverings, wraps, stickers, etc.) sold under the trade names of Jamberry® or Jamberry Nails®.

As used herein, the terms “removal time,” “removal rate,” and similar terms refer to the duration of time for a wearer of a nail coating to expose the nail coating to the nail coating removal composition and wipe, peel, lift, or otherwise remove the nail coating completely from the nail plate. In situations where the nail coating is a solid nail coating, such as a decorative adhesive nail film or strip, the removal time represents the amount of time taken for a user to contact the nail coating and nail with the nail coating removal composition and to peel, lift, or slide the nail coating from the nail plate without resistance and without damaging the nail plate or surrounding tissues.

As used herein, the term “natural oil” and similar terms refer to various plant-based oils including, but not limited to, cottonseed, flaxseed, grapeseed, coconut (typically labeled as 76°, 91°, 92°, etc., referring to the melting point of the oil in degrees F., and also commonly available in fractionated form with a higher proportion of C8 and C10 fatty acids), avocado, argan, babussu, hempseed, corn, safflower, poppyseed, sunflower, walnut, soya bean, canola, chia seed, perilla seed, palm kernel (flakes or oil), peanut, palm, jojoba, macadamia nut, sesame seed, meadowfoam seed, wheat germ, and olive oils. Other plant-based oils suitable for safe application to the nail plate and surrounding tissues are also included within this definition.

The term “natural oil” and similar terms also include extracted or purified saturated and unsaturated fatty acids, particularly fatty acids within the C8 to C18 range, such as caprylic acid (C8), capric acid (C10), lauric acid (C12), myristic acid (C14), palmitic acid (C16), stearic acid (C18), palmitoleic acid (C16:1), oleic acid (C18:1), linoleic acid (C18:2), linolenic acid (C18:3), and stearidonic acid (C18:4), for example.

As used herein, the term “natural oil” and similar terms also include alcohols of fatty acids (e.g., a natural reduction product of one or more fatty acids), such as capryl alcohol (C8), capric alcohol (C10), lauryl alcohol (C12), myristyl alcohol (C14), cetyl alcohol (C16), stearyl alcohol (C18), and palmitoleyl alcohol (C16:1), for example.

The terms “natural oil blend” or “oil component” refer to an amount of a natural oil, fatty acid, or fatty alcohol in pure form or in combination with one or more additional natural oils and/or other additives.

The terms “additives,” “additional ingredients,” “added components,” and the like can include antioxidants (e.g., Tocopherol) or other preservatives, skin or nail conditioning agents, surfactants, emulsifiers, carrier agents, mono-, di-, or tri-glycerides, glycerol, colorants, fragrances, and/or other beneficial ingredients.

Alkyl esters of fatty acids may also be included as additives; however, in preferred embodiments, the amount of fatty acid alkyl esters is less than 50%, less than 25%, less than 10%, less than 5%, or less than 1% by weight of the natural oil blend or of the overall composition. For example, preferred embodiments include only those fatty acid alkyl esters that occur naturally in the oils of the composition without chemical modification.

As used herein, the terms “volatile solvent,” “volatile solvent-like substance,” and similar terms refer to a compound or mixture of compounds that are volatile and/or that tend to penetrate into the upper layers of the nail plate. The foregoing terms also refer to compositions that which tend to strip away beneficial oils from the nail plate and/or surrounding tissues. As used herein, “volatile solvent” includes short-chain alcohols, ketones, and alkyl esters of organic acids (e.g., alkyl esters of carboxylic acids such as methyl acetate, ethyl acetate, methyl lactate, ethyl lactate, etc.). As explained in further detail below, such compounds are preferably omitted from the nail removal compositions described herein, or are included in relatively small amounts.

As used herein, the terms “conditioning,” “regenerating,” “protecting,” and similar terms, as applied to the nail plate and/or surrounding tissues, refer to activity that promotes good tissue health for the associated tissues. For example, a nail coating removal composition may prevent the thinning, and/or reduce the displacement (e.g., flaking, peeling) of upper layer cells of the nail plate. Such terms can refer to activity that prevents or reduces dehydration or brittleness of the nail plate (e.g., by preventing or reducing the stripping away of beneficial oils and moisture associated with the nail plate).

Unless specifically described otherwise, all percentages and other ratios are expressed as % wt. or as ratios based on relative weights of the components or sub-components of the nail coating removal compositions.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition. In addition, unless expressly described otherwise, all stated amounts (e.g., concentrations, percentages, ratios, etc.) are to be interpreted as being “approximately,” “about,” and/or “substantially” the stated amount, regardless of whether the terms “approximately,” “about,” and/or “substantially” are expressly stated in relation to the stated amount(s).

### Nail Coating Removal Compositions

In some embodiments, a nail coating removal composition includes a natural oil blend that is substantially free of solvents such as short-chain alcohols, ketones, esters, and other oil dissolving substances. For example, a nail coating removal composition may have less than 30%, less than 20%, less than 10%, less than 5%, less than 2.5%, less than 1%, less than 0.1%, or less than 0.01% by weight of short-chain alcohols, ketones, alkyl esters of organic acids, or other solvents, or may have non-detectable levels of such substances.

It has been found that nail coating removal compositions of the present disclosure that reduce or eliminate volatile solvent and volatile solvent-like substances are able to beneficially protect the nail plate, aid in the regeneration of damaged nail plates and other nail tissue, and provide the nail plate with greater resistance to future damage. In addition, when nail coating removal compositions are formulated according to one or more embodiments disclosed herein, the removal composition can provide such benefits to nail tissues while also functioning to effectively remove the nail coating.

Similarly, nail coating removal compositions preferably omit substances that are insoluble in the natural oil blend or that tend to separate from the solution (e.g., urea and/or urea-based compounds). For example, in some embodiments, compounds having low solubility within the natural oil blend of the removal composition are omitted or are included at reduced levels in order to prevent changes to the consistency of the composition, to prevent the formation of a crystalline product, and/or to prevent formation of a precipitate (e.g., at room temperature or about 21 degrees C.), inhibiting the effectiveness of the composition. For example, many free fatty acids and/or oils can crystalize or form precipitates if included in a concentration that is too high (for a particular temperature). One or more embodiments are able to provide benefits to the condition of the nail and/or skin tissues and are able to provide effective removal of the nail coating without the detrimental effects (e.g., to appearance, stability, storability, and/or consistency) of insoluble substances.

The natural oil blend includes one or more natural oils selected to enhance the texture, appearance, and/or tissue effects (e.g., nail/skin tissue conditioning, protecting, and/or regeneration) of the nail coating removal composition. As explained in greater detail below, it has been found that the natural oil blends disclosed herein enable enhanced removal of nail coatings with reduced or eliminated detrimental effects to the nail plate, and at least in some circumstances with enhanced regenerative effects to the nail plate (e.g., increases in flexibility, durability, toughness, etc.). In some embodiments, a combination of different natural oils functions to synergistically reduce nail coating removal times.

In preferred embodiments, the natural oil blend is formed of one or more of cottonseed oil, flaxseed oil, hempseed oil, chia seed oil, pumpkinseed oil, grapeseed oil, perilla seed oil, wheat germ oil, safflower oil, and poppyseed oil. Some embodiments include a natural oil in combination with a caprylic acid, myristic acid, myristic alcohol, cetyl alcohol, stearyl alcohol, stearic acid, oleic acid linoleic acid, and/or lauric acid component, which may be added to make up about 0.5 to 40%, or about 1 to 30%, or about 2 to 20% of the overall composition (e.g., may be added to one or more of the foregoing natural oils).

These components, and blends including one or more of these components, have been found to particularly enhance the removal rate of a nail coating removal composition and/or particularly enhance the nail protecting and regenerating properties when included. For example, lipid and free fatty acid regeneration can prevent cracking of the skin surrounding the nail plate, as well as peeling, splitting, and cracking of the nail plate, and the condition of the nail plate of a user can thereby improve with each use of a removal composition. Testing has shown that users see improvements to flexibility and toughness/durability of the nail plate. Surprisingly, at least some of the natural oil blends as described herein were found to exhibit beneficial synergistic effects when the separate components were combined to form the removal compositions, as compared to the functionality of the components when used independently.

In some embodiments, the natural oil blend has a high concentration of C12 to C18 fatty acids and/or fatty acid alcohols. In preferred embodiments, the natural oil blend has a high concentration of C16 to C18 fatty acids and/or fatty acid alcohols, and more particularly a high concentration of C18 fatty acids and/or fatty acid alcohols. In one example, the natural oil blend may be formulated with a fatty acid profile of about 90% or more, or about 95% or more, or about 97% or more of C12 to C18 fatty acids and/or fatty acid alcohols. In another example, the natural oil blend is formulated with a fatty acid profile of about 90% or more, or about 93% or more, or about 95% or more, or about 97% or more of C16 to C18 fatty acids and/or fatty acid alcohols. In another example, the natural oil blend is formulated with a fatty acid profile of about 60-95%, or about 65-90%, or about 70-85% of C18 fatty acids and/or fatty acid alcohols. In some embodiments, the natural oil blend has a C18 fatty acid profile of greater than about 65%, 70%, 75%, 80%, 85%, 90%, or 95%.

### Mode of Action

Surprisingly, it has been found that one or more embodiments disclosed herein are able to provide effective removal of nail coatings while simultaneously reducing or eliminating the detrimental effects associated with traditional nail coating removal compositions (e.g., drying of nail plate and surrounding tissues, peeling and cracking of edge of nail plate, etc.).

Without being bound to any particular theory, it is thought that one or more components of the composition function to weaken or degrade the interface between the nail plate and the nail coating (e.g., by disrupting the external adhesion of the covering to the nail plate, such as by disrupting the adhesive attaching the covering to the nail plate). This functionality beneficially enhances the removal rate of the composition, leading to more effective and/or less-damaging removal and greater rates of user compliance with proper removal techniques. Users are therefore more likely to follow proper procedures during nail coating removal, as opposed to peeling or pulling too forcefully or too rapidly during removal and causing trauma to the nail plate (causing or increasing the risk of nail plate pitting, flaking, pealing, spotting, and/or splitting).

For example, in applications directed to the removal of a solid nail coating, the adhesive used to bind the solid covering to the nail plate has been found to exhibit a degree of structural porosity. Embodiments of nail removal compositions have been found to be capable of wicking into the internal matrix of the binding adhesive (e.g., through capillary action actuated by the porous structure of the adhesive matrix). By wicking or otherwise migrating into the interstitial space between the nail coating and the nail plate, the oils function to bring the removal composition into contact with the adhesive to disrupt the adhesion between the nail coating and the nail plate.

In some embodiments, the nail removal compositions have been found to soften the adhesive such that it becomes more flexible and more susceptible to being frayed, ruptured, and/or severed upon subjection to a removal force. In this manner, pulling or peeling forces applied to the nail plate are relieved by the disruption of the adhesive rather than by raising or detaching of cells at the upper layers of the nail plate.

Beneficially, this wicking function more rapidly brings the removal composition into contact with the adhesive at all areas along the interface between the nail coating and nail plate, as opposed to merely contacting the adhesive at or near the edges of the interface or only moving inwards as the nail coating is progressively removed to expose a greater portion of the nail plate. This action can lead to faster removal of the nail coating. In some embodiments, separate components of the natural oil blend have been found to synergistically function together to provide faster contact with and disruption of the adhesive.

In preferred embodiments, the nail coating removal composition omits fatty acid alkyl esters. For example, in applications for removal of a solid nail coating, a natural oil blend component having a greater proportion of non-esterified fatty acids (i.e., free fatty acids that maintain the carboxylic acid group, and are not covalently linked to another fatty acid molecule) to fatty acid esters has been found to provide greater wicking of the composition into the matrix of the adhesive at the interface between the nail plate and the nail coating.

In some embodiments, a removal composition is capable of weakening or degrading the cohesion of the nail coating itself. For example, in certain applications where the nail coating is a solid nail coating, the removal composition has been found to weaken the integrity of the solid nail coating. In some circumstances, this can accelerate wicking and enables easier removal of the solid nail coating from the nail plate (e.g., by allowing a user to more easily bend, fold, or otherwise manipulate the covering as it is peeled or lifted off the nail plate).

In preferred embodiments, insoluble constituents and constituents with a tendency to crystalize or separate out of solution are omitted or reduced to levels at which crystallization and precipitate formation are negligible. The reduction or omission of such substances is beneficial for a number of reasons. For example, avoiding the formation of crystalized products and/or solid precipitates avoids potential hampering of the advantageous wicking effect described above (e.g., as a result of precipitate blocking passage of the remover through the adhesive matrix or otherwise adjusting the fluid dynamics of the removal process) and avoids the formation of crystals and/or precipitate during storage or while the composition is in a packaging container, lowering the efficacy of the composition.

### Methods of Use

In some embodiments, a method of removing a nail coating from a nail plate of a wearer includes: (1) providing a nail coating removal composition, the nail coating removal composition including one or more natural oils and being substantially free of volatile solvents; (2) contacting the nail coating removal composition to a nail coating; and (3) removing the nail coating from the nail plate.

In some embodiments, a method of removing a nail coating from a nail plate further includes, after contacting the nail coating removal composition to the nail coating, allowing the nail coating removal composition to disrupt at least one of the cohesive integrity of the nail coating and/or the adhesive bond between the nail coating and the nail plate. In some embodiments, the nail coating is a solid nail coating, such as a nail strip, wrap or film, and the nail coating removal composition enables removal by disrupting the cohesive integrity of the nail coating itself and/or by disrupting the adhesive bond between the nail coating and the nail plate (e.g., through wicking action of one or more of the natural oils included in the composition).

The nail coating removal methods described herein beneficially enable effective removal without exacerbating detrimental effects to the nail plate and/or surrounding tissues. In addition, the effective removal with simultaneous nail conditioning can increase nail durability and user compliance with proper removal protocol, as opposed to causing premature removal with associated nail plate damage.

### EXAMPLES

### Example 1

Solid nail coatings with adhesive layers formed from an acrylate(s)/ethylhexyl acrylate copolymer were affixed (by pressure-sealing and heat-sealing) lengthwise to 0.75 inch diameter round glass tubes. Before adhering each nail coating to its respective tube, a thin nylon wire was passed transversely across and under the width of the nail coating. The nylon wire was then passed out of the center of the nail coating so that the tube could be suspended (and balanced evenly) by the nylon wire. The tubes were filled with steel beads to a weight of 100 grams+/−0.01 grams. Each tube was then submerged into a bath containing a nail coating removal composition, and a timer measured the length of time for the tube to break free and drop to the bottom of the bath. Times ranged from between 1 to 6 minutes. Compositions including coconut, grapeseed, cottonseed, and/or flaxseed gave the best results.

### Example 2

Nail removal compositions were tested on solid nail coatings (having acrylate(s)/ethylhexyl acrylate copolymer adhesive layers) attached to several wearer's nails by pressure and heat sealing. Typically, after 3 to 7 days of wear, users dipped a cotton pad into the remover composition, and then rubbed the wetted pad over the top of the nail coating for a time sufficient to wet the entire nail coating. Then using the saturated pad and a fingernail, the wearer caught an edge of the coating that had begun to loosen and then gently worked the exposed underside backward with the wetted pad and their finger, progressively exposing more and more of the underside of the solid coating material. Compositions including coconut, grapeseed, cottonseed, and/or flaxseed gave the best results. Accelerated removal was also seen when users soaked a fingernail in a removal composition prior to removal (as opposed to application with the cotton pad).

### Example 3

Various formulations including urea were tested and removal times were recorded following the removal procedure described in Example 2, with removal being initiated after 3-5 days of wear. Five users were tested, and each separate formulation was used on 6-10 different fingers (no more than two fingers on the same user). The solid nail coatings were adhered by heat-sealing and pressure-sealing an acrylates/ethylhexyl acrylate copolymer adhesive layer to the respective nail plate). The tested compositions included varying amounts of urea and lauric acid. Results are shown in Table 1.

TABLE 1

| | | Average Removal Time |
|---|---|---|
| % wt. lauric acid | % wt. urea | (seconds) |
| 0 | 0 | 33 |
| 40 | 0 | 19 |
| 0 | 7 | 28 |
| 20 | 0 | 20 |
| 0 | 3.5 | 25 |
| 20 | 3.5 | 26 |

As shown in Table 1, compositions including lauric acid and omitting urea performed better than those having urea without lauric acid or those having urea and lauric acid. In the composition including both lauric acid and urea, it is likely that the urea formed chemical complexes with the lauric acid (urea being known to complex with fatty acids; see, e.g., Progress in the chemistry of fats and other lipids, Vol. 2, Pergamon Press, NY, 1954, pp. 243-267; Swern et al., J. Am. Oil Chem. Soc. 1952, Vol. 29:10, pp. 431-434), neutralizing the effectiveness of both components as a removal accelerator.

### Example 4

Various formulations having different levels of methyl laurate were tested. Results are shown in Table 2. Notably, the compositions with methyl laurate failed to outperform the control. It is theorized that removal of the carboxylic acid end group from the carbon backbone has a detrimental effect on removal efficiency, and that the acidic functionality can provide key benefits.

TABLE 2

| | Average removal time |
|---|---|
| % wt. Methyl Laurate | (seconds) |
| 0 | 25 |
| 10 | 33 |
| 20 | 29 |
| 30 | 36 |
| 40 | 39 |

### Example 5

Various formulations having different levels of methyl oleate were tested. One user tested each formulation on 1-2 different fingers. Results are shown in Table 3. Notably, the compositions with methyl oleate failed to outperform the control. It is theorized that removal of the carboxylic acid end group from the carbon backbone has a detrimental effect on removal efficiency, and that the acidic functionality provides key benefits.

TABLE 3

| | Average removal time |
|---|---|
| % wt. Methyl Oleate | (seconds) |
| 0 | 9 |
| 10 | 24 |
| 20 | 12 |
| 30 | 10 |
| 40 | 19 |

### Examples 6-9

In Examples 6-9, various different formulations were tested, and corresponding removal times were recorded, following the removal procedure described in Example 2, with removal being initiated after 3-5 days of wear. The solid nail coatings were adhered by heat-sealing and pressure-sealing an acrylate(s)/ethylhexyl acrylate copolymer adhesive layer to the respective nail plate.

### Example 6

Several different natural oils were tested for removal times. Results are shown in Table 4.

TABLE 4

| | Avg. removal time | |
|---|---|---|
| Natural Oil | (seconds) | Number of users (n) |
| Cottonseed | 38 | ~280 |
| Grapeseed | 56 | 4 |
| Flaxseed | 44 | 4 |
| Wheat germ | 41 | 5 |
| Pumpkinseed | 37 | 5 |
| Chia seed | 44 | 4 |
| Hempseed | 52 | 4 |
| Oleic acid | 49 | 9 |
| Linoleic acid | 51 | 9 |
| Linolenic acid | 49 | 9 |
| Safflower oil | 38 | 5 |
| Poppyseed oil | 35 | 5 |

### Example 7

Various natural oil blends made using a cottonseed oil base were compared against a pure cottonseed oil standard, and average reductions in removal times relative to the cottonseed oil standard were measured. Results are shown in Table 5.

TABLE 5

| Composition | Formulation (in | Avg. reduction in | Number of users |
|---|---|---|---|
| No. | cottonseed oil base) | removal time | (n) |
| 1 | 2% Caprylic acid | 10% | 7 |
| 2 | 4% Myristic acid | 6% | 7 |
| 3 | 2% Myristic acid | 16% | 7 |
| 4 | 0.66% Myristic acid | No reduction | 7 |
| 5 | 2% Myristic alcohol | No reduction | 9 |
| 6 | 2% Cetyl alcohol | No reduction | 9 |
| 7 | 2% Stearyl alcohol | 29% | 9 |
| 8 | 2% Stearic acid | 19% | 9 |
| 9 | 20% Oleic acid | 9% | 6 |
| 10 | 20% Linoleic acid | 4% | 6 |
| 11 | 10% Lauric acid | 30% | 7 |
| 12 | 6% Lauric acid | 16% | 7 |
| 13 | 4% Lauric acid | 13.5% | 7 |
| 14 | 30% Hempseed oil | 20% | 7 |
| 15 | 2% Babussu oil | 14% | 7 |
| 16 | 2% Coconut oil | No reduction | 7 |
| 17 | 2% Lauric acid and | No reduction | 4 |
| | 2% Palmitic acid | | |
| 18 | 4% Palmitic acid | No reduction | 4 |
| 19 | 10% Wheat germ oil | 15% | 7 |
| 20 | 20% Wheat germ oil | 29% | 7 |
| 21 | 30% Wheat germ oil | 11% | 7 |
| 22 | 40% Wheat germ oil | 3% | 7 |
| 23 | 1.5% Behenyl | 13% | 7 |
| | alcohol | | |
| 24 | 1.5% C32-C36 | No reduction | 7 |
| | alcohols | | |

### Example 8

Nail coating removal compositions were formulated by mixing the components shown in Table 6. These nail coating compositions are readily applied to nail coatings to effect removal of the nail coatings.

TABLE 6

| Composition No. | Component | % weight |
|---|---|---|
| 25 | Cottonseed oil | 65 |
| | Wheat germ oil | 32 |
| | Lauric acid | 3 |
| 26 | Cottonseed oil | 65 |
| | Wheat germ oil | 29 |
| | Lauric acid | 3 |
| | Stearic acid | 3 |
| 27 | Cottonseed oil | 65 |
| | Wheat germ oil | 26 |
| | Stearic acid | 3 |
| | Stearyl alcohol | 3 |
| 28 | Cottonseed oil | 65 |
| | Wheat germ oil | 15 |
| | Hempseed oil | 14 |
| | Lauric acid | 3 |
| | Stearic acid | 3 |

### Example 9

Additional nail coating removal compositions were formulated by mixing the compositions shown in Table 7. The nail coating removal compositions were readily applied to nail coatings to effect removal of the nail coatings.

TABLE 7

| Composition No. | Component | % weight |
|---|---|---|
| 29 | Coconut Oil 91° | 58 |
| | Flaxseed Oil | 32 |
| | Grapeseed Oil | 8.5 |
| | Jojoba oil | 1 |
| | Tocopherol | 0.5 |
| 30 | Cottonseed Oil (RBDW) | 56 |
| | Flaxseed Oil | 31 |
| | Grapeseed Oil | 8.1 |
| | Cetyl Alcohol | 4.9 |
| 31 | Cottonseed Oil (RBDW) | 56 |
| | Flaxseed Oil | 31 |
| | Grapeseed Oil | 6.5 |
| | Cetyl Alcohol | 6.5 |
| 32 | Cottonseed oil (RBDW) | 57.9 |
| | Flaxseed oil | 32.2 |
| | Grapeseed | 8.5 |
| | Tocopherol | .3 |
| | Jojoba oil | 1.1 |
| 33 | Cottonseed oil (RBDW) | 64.0 |
| | Caprylic/Capric acid | 23.6 |
| | Myristic acid | 6.2 |
| | Lauric acid | 6.2 |
| 34 | Caprylic/Capric Acid | 66.2 |
| | Lauric acid | 17.8 |
| | Myristic Acid | 16.0 |
| 35 | Babussu oil | 63.3 |
| | Cottonseed oil | 34.7 |
| | Myristic acid | 2 |
| 36 | Sunflower oil | 89.9 |
| | Lauric acid | 6.0 |
| | Cetyl Alcohol | 4.1 |
| 37 | Grapeseed oil | 68.7 |
| | Monolaurin 95% | 31.4 |
| 38 | Grapeseed oil | 30.1 |
| | Coconut Oil 91° | 30.1 |
| | Flaxseed oil | 29.7 |
| | Met 10U | 10.1 |
| 39 | Myristic acid | 9.3 |
| | Caprylic/Capric Acid | 90.7 |
| 40 | Babussu oil | 44.8 |
| | Flaxseed oil | 44.8 |
| | Caprylic/Capric Acid | 10.4 |

### Example 10

Fatty acid profiles for various removal compositions listed in Examples 7-9 were calculated using the fatty acid profile information listed in Table 8 (fatty acid amounts of oils listed as % wt.).

TABLE 8

| | Linolenic | Linoleic | Oleic | Stearic | Palmitic | Myristic | Lauric | Capric | Caprylic |
|---|---|---|---|---|---|---|---|---|---|
| Oil | C18:3 | C18:2 | C18:1 | C18 | C16 | C14 | C12 | C10 | C8 |
| Cottonseed | | 53 | 21 | 4.5 | 21.5 | | | | |
| Flaxseed | 35-60 | 17-24 | 12-34 | 2-5 | 4-7 | | | | |
| Grapeseed | 0.1 | 70 | 16 | 4 | 7 | | | | |
| Coconut 91° | | | 5-8 | 1.3 | 7.5-10.5 | 13-19 | 44-52 | 4.5-9.5 | 5.5-9.5 |
| Coconut | | | | | | | | 27.6 | 72.3 |
| (fractionated) | | | | | | | | | |
| Babussu | | | 12-18 | | 6-9 | 15-20 | 44-46 | 2.7-7.6 | 4-6.5 |
| Hempseed | 24-26 | 54-56 | 11-13 | 1-3 | 5-7 | | | | |
| Corn | | 34-62 | 19-49 | 2-5 | 8-12 | 0.2-1 | | 7 | 4 |
| Safflower | | 73-79 | 13-21 | 1-4 | 3-6 | | | | |
| Poppyseed | 5 | 72 | 11 | 2 | 10 | | | | |
| Sunflower | | 44-75 | 14-35 | 1-3 | 3-6 | | | | |
| Walnut | 14 | 58 | 13 | | | | | | |
| Soya bean | 5-11 | 43-56 | 22-34 | 2-6 | 7-11 | | | | |
| Canola | 8.6 | 20 | 63 | 2 | 4 | | | | |
| Chia seed | 62 | 17 | 11 | 2.67 | 6.69 | | | | |
| Perilla | 62-65 | 13-15 | 12-14 | 1-3 | 6-8 | | | | |
| Palm Kernel | | 0.5-2 | 11-19 | 1-3 | 7-9 | 14-18 | 40-52 | 3-7 | 3-5 |
| Palm | | 5-11 | 38-52 | 2-7 | 32-45 | 0.5-2 | | | |
| Olive | | 4-10 | 65-80 | 1-3 | 7-16 | .1-1.2 | | | |
| Wheat germ | 4-10 | 55-60 | 13-21 | 2 | 13-20 | | | | |
| Pumpkin seed | | 64 | 11 | 9.9 | 14 | | | | |
| Camellia | | 7-10 | 80-88 | 1-2 | 7-9 | | | | |
| Evening | 8-14 | 65 | 6-11 | 2 | 6-7 | | | | |
| primrose | | | | | | | | | |
| Rosehip seed | 29-36 | 41-49 | 29-36 | 1-3 | 3-5 | | | | |

The fatty acid distribution (in % wt. of the composition) of different fatty acids and the distribution within various ranges (e.g., C8 to C18) of various removal compositions are shown in Tables 9 and 10, respectively. The data shown in Table 8 can also be used to calculate the fatty acid profiles of other formulations described herein and/or to formulate other natural oil removal compositions based on a desired fatty acid profile.

TABLE 9

| Composition | | | | | C18 | | | | | |
|---|---|---|---|---|---|---|---|---|---|---|
| No. | C18:3 | C18:2 | C18:1 | C18:0 | (all) | C16 | C14 | C12 | C10 | C8 |
| 1 | 0 | 52 | 21 | 4 | 77 | 21 | 0 | 0 | 0 | 2 |
| 2 | 0 | 51 | 20 | 4 | 75 | 21 | 4 | 0 | 0 | 0 |
| 3 | 0 | 52 | 21 | 4 | 77 | 21 | 2 | 0 | 0 | 0 |
| 7 | 0 | 52 | 21 | 6 | 79 | 21 | 0 | 0 | 0 | 0 |
| 8 | 0 | 52 | 21 | 6 | 79 | 21 | 0 | 0 | 0 | 0 |
| 9 | 0 | 42 | 37 | 4 | 83 | 17 | 0 | 0 | 0 | 0 |
| 11 | 0 | 48 | 19 | 4 | 71 | 19 | 0 | 10 | 0 | 0 |
| 12 | 0 | 50 | 20 | 4 | 74 | 20 | 0 | 6 | 0 | 0 |
| 13 | 0 | 51 | 20 | 4 | 75 | 21 | 0 | 4 | 0 | 0 |
| 14 | 8 | 54 | 18 | 4 | 83 | 17 | 0 | 0 | 0 | 0 |
| 15 | 0 | 52 | 21 | 4 | 77 | 21 | 0 | 1 | 0 | 0 |
| 19 | 1 | 53 | 21 | 4 | 79 | 21 | 0 | 0 | 0 | 0 |
| 20 | 1 | 54 | 20 | 4 | 80 | 21 | 0 | 0 | 0 | 0 |
| 21 | 2 | 54 | 20 | 4 | 80 | 20 | 0 | 0 | 0 | 0 |
| 22 | 3 | 55 | 19 | 4 | 81 | 20 | 0 | 0 | 0 | 0 |
| 25 | 2 | 53 | 19 | 4 | 78 | 19 | 0 | 3 | 0 | 0 |
| 26 | 2 | 51 | 19 | 7 | 78 | 19 | 0 | 3 | 0 | 0 |
| 27 | 2 | 49 | 18 | 9 | 79 | 18 | 0 | 0 | 0 | 0 |
| 28 | 5 | 51 | 18 | 7 | 80 | 17 | 0 | 3 | 0 | 0 |

TABLE 10

| Composition | C8- | C8- | C8- | C8- | C8- | C10- | C10- | C10- |
|---|---|---|---|---|---|---|---|---|
| No. | C10 | C12 | C14 | C16 | C18 | C12 | C14 | C16 |
| 1 | 2 | 2 | 2 | 23 | 100 | 0 | 0 | 21 |
| 2 | 0 | 0 | 4 | 25 | 100 | 0 | 4 | 25 |
| 3 | 0 | 0 | 2 | 23 | 100 | 0 | 2 | 23 |
| 7 | 0 | 0 | 0 | 21 | 100 | 0 | 0 | 21 |
| 8 | 0 | 0 | 0 | 21 | 100 | 0 | 0 | 21 |
| 9 | 0 | 0 | 0 | 17 | 100 | 0 | 0 | 17 |
| 11 | 0 | 10 | 10 | 29 | 100 | 10 | 10 | 29 |
| 12 | 0 | 6 | 6 | 26 | 100 | 6 | 6 | 26 |
| 13 | 0 | 4 | 4 | 25 | 100 | 4 | 4 | 25 |
| 14 | 0 | 0 | 0 | 17 | 100 | 0 | 0 | 17 |
| 15 | 0 | 1 | 1 | 23 | 100 | 1 | 1 | 23 |
| 19 | 0 | 0 | 0 | 21 | 100 | 0 | 0 | 21 |
| 20 | 0 | 0 | 0 | 21 | 100 | 0 | 0 | 21 |
| 21 | 0 | 0 | 0 | 20 | 100 | 0 | 0 | 20 |
| 22 | 0 | 0 | 0 | 20 | 100 | 0 | 0 | 20 |
| 25 | 0 | 3 | 3 | 22 | 100 | 3 | 3 | 22 |
| 26 | 0 | 3 | 3 | 22 | 100 | 3 | 3 | 22 |
| 27 | 0 | 0 | 0 | 18 | 97 | 0 | 0 | 18 |
| 28 | 0 | 3 | 3 | 20 | 100 | 3 | 3 | 20 |

| Composition | C10- | C12- | C12- | C12- | C14- | C14- | C16- |
|---|---|---|---|---|---|---|---|
| No. | C18 | C14 | C16 | C18 | C16 | C18 | C18 |
| 1 | 98 | 0 | 21 | 98 | 21 | 98 | 98 |
| 2 | 100 | 4 | 25 | 100 | 25 | 100 | 96 |
| 3 | 100 | 2 | 23 | 100 | 23 | 100 | 98 |
| 7 | 100 | 0 | 21 | 100 | 21 | 100 | 100 |
| 8 | 100 | 0 | 21 | 100 | 21 | 100 | 100 |
| 9 | 100 | 0 | 17 | 100 | 17 | 100 | 100 |
| 11 | 100 | 10 | 29 | 100 | 19 | 90 | 90 |
| 12 | 100 | 6 | 26 | 100 | 20 | 94 | 94 |
| 13 | 100 | 4 | 25 | 100 | 21 | 96 | 96 |
| 14 | 100 | 0 | 17 | 100 | 17 | 100 | 100 |
| 15 | 100 | 1 | 22 | 100 | 22 | 99 | 98 |
| 19 | 100 | 0 | 21 | 100 | 21 | 100 | 100 |
| 20 | 100 | 0 | 21 | 100 | 21 | 100 | 100 |
| 21 | 100 | 0 | 20 | 100 | 20 | 100 | 100 |
| 22 | 100 | 0 | 20 | 100 | 20 | 100 | 100 |
| 25 | 100 | 3 | 22 | 100 | 19 | 97 | 97 |
| 26 | 100 | 3 | 22 | 100 | 19 | 97 | 97 |
| 27 | 97 | 0 | 18 | 97 | 18 | 97 | 97 |
| 28 | 100 | 3 | 20 | 100 | 17 | 97 | 97 |

The described embodiments are to be considered in all respects only as illustrative and not restrictive. The scope of the disclosure is, therefore, indicated by the appended claims rather than by the foregoing description. Elements described in relation to any embodiment described herein may be combined with elements described in relation to any other embodiment described herein.

1. A nail coating removal composition, comprising a blend of natural oils formulated to promote removal of a nail coating from a nail plate upon contact with the nail coating and formulated to condition the nail plate, wherein the removal composition is substantially free of volatile solvents.

2. The composition of claim 1, wherein the nail coating composition is formulated to remove a solid nail coating.

3. The composition of claim 1, wherein the nail coating composition is formulated to avoid damaging a nail plate to which it is contacted.

4. The composition of claim 1, wherein the natural oil blend is formulated from plant-based oils.

5. The composition of claim 1, wherein the natural oil blend is formulated from the group consisting of: cottonseed, flaxseed, grapeseed, coconut, avocado, argan, babussu, hempseed, corn, safflower, poppyseed, sunflower, walnut, soya bean, canola, chia seed, perilla seed, palm kernel, peanut, palm, jojoba, macadamia nut, sesame seed, meadowfoam seed, pumpkin seed, wheat germ, and olive oil; caprylic, capric, lauric, myristic, palmitic, stearic, palmitoleic, oleic, linoleic, linolenic, and stearidonic acid, and fatty acid alcohols thereof.

6. The composition of claim 1, wherein an amount of fatty acid alkyl esters is less than 10% by weight of the composition.

7. The composition of claim 1, wherein fatty acid alkyl esters are omitted.

8. The composition of claim 1, wherein substances insoluble in the removal composition at room temperature are omitted.

9. The composition of claim 1, wherein one or more natural oils of the removal composition are formulated to wick into a matrix of an adhesive to weaken and/or soften the adhesive.

10. The composition of claim 1, wherein the blend of natural oils includes one or more of cottonseed oil, hempseed oil, wheat germ oil, chia seed oil, flaxseed oil, grapeseed oil, perilla seed oil, or pumpkinseed oil.

11. The composition of claim 1, wherein the blend of natural oils includes one or more of a lauric acid, caprylic acid, myristic acid, stearic acid, oleic acid, linoleic acid, myristic alcohol, cetyl alcohol, and stearyl alcohol component at about 1 to 30% of the composition.

12. The composition of claim 1, wherein the blend of natural oils includes one or more of a lauric acid, caprylic acid, myristic acid, stearic acid, oleic acid, linoleic acid, myristic alcohol, cetyl alcohol, and stearyl alcohol component at about 2 to 20% of the composition.

13. The composition of claim 1, wherein the blend of natural oils has a fatty acid profile of about 90% or more of C16 to C18 fatty acids or fatty alcohols.

14. The composition of claim 1, wherein the blend of natural oils has a fatty acid profile of about 95% or more of C16 to C18 fatty acids or fatty alcohols.

15. The composition of claim 1, wherein the blend of natural oils has a fatty acid profile of about 65% or more of C18 fatty acids or fatty acid alcohols.

16. The composition of claim 1, wherein the blend of natural oils has a fatty acid profile of about 70% or more of C18 fatty acids or fatty acid alcohols.

17. A nail coating removal composition, comprising a blend of natural oils formulated to promote removal of a nail coating from a nail plate upon contact with the nail coating and formulated to condition the nail plate,
wherein the removal composition is substantially free of volatile solvents, and
wherein the natural oil blend has a fatty acid profile of about 90% or more of C16 to C18 fatty acids or fatty alcohols.

18. The composition of claim 17, wherein the natural oil blend has a fatty acid profile of about 65% or more of C18 fatty acids or fatty acid alcohols.

19. A method of removing a nail coating from a nail plate of a wearer, the method comprising:
providing a nail coating removal composition including a blend of natural oils formulated to promote removal of a nail coating from a nail plate upon contact with the nail coating and formulated to condition the nail plate, wherein the removal composition is substantially free of volatile solvents, and wherein the blend of natural oils has a fatty acid profile of about 65% or more of C18 fatty acids or fatty acid alcohols;
contacting the nail coating removal composition to a nail coating; and
removing the nail coating from the nail plate.

20. The method of claim 19, wherein the blend of natural oils includes one or more of a lauric acid, caprylic acid, myristic acid, stearic acid, oleic acid, linoleic acid, myristic alcohol, cetyl alcohol, and stearyl alcohol component at about 2 to 20% of the composition.
